(19) United States
(12) Patent Application Publication
(10) Pub. No.: US 2008/0228278 A1
(43) Pub. Date: Sep. 18, 2008

(54) Modular Prosthesis For Mandibular Reconstruction

(75) Inventors: Shermin Lee, Singapore (SG); Henk Tideman, Singapore (CN)

Correspondence Address: HARNESS, DICKEY & PIERCE, P.L.C., P.O. BOX 828, BLOOMFIELD HILLS, MI, 48303, US

(73) Assignee: BIOMET MICROFIXATION INC., Jacksonville, FL (US)

(21) Appl. No.: 12/126,269

(22) Filed: May 23, 2008

Related U.S. Application Data
(63) Continuation of application No. PCT/SG2005/000402, filed on Jun. 12, 2006.

(51) Int. Cl. A61F 2/28

(52) U.S. Cl. 623/17.17

(57) ABSTRACT

A prosthetic module for a mandible bone includes a body having a form corresponding to the mandible bone and extending between a first end and a second end positioned opposite the first end. A first stem extends from the body at the first end and is configured to extend into and couple the body to a mandible bone in replacement of a resected section thereof. At least one of a second stem, a hole, or a geometry similar to a condyle-ramus-angle part of a mandible bone is located at the second end thereof.

### CROSS-REFERENCE TO RELATED APPLICATIONS

This application is a continuation of International Application No. PCT/SG2005/000402, filed Nov. 25, 2005. The disclosure of the above application is incorporated herein by reference.

### FIELD

The present disclosure relates to a prosthesis for the reconstruction of a lower jaw or mandible that has been rendered segmental as a result of resection of a diseased or damaged part of the mandible. The prosthesis includes a plurality of prosthetic modules which may be chosen to join to one another or to bridge the resection of the segmental mandible.

### BACKGROUND

The statements in this section merely provide background information related to the present disclosure and may not constitute prior art.

A lower jaw or mandible may require a resection, which is defined as partial surgical removal, due to diseases such as cancer (e.g., squamous cell carcinoma, intra-osseous carcinoma), aggressive benign tumor, osteomyelitis, radio therapy, infection, etc. The resected mandible is termed segmental, i.e., comprising 2 disjointed segments due to loss in bone continuity. Mandibular reconstruction therefore strives to restore the patient's resected mandible to an appearance as natural as possible including implantation of teeth on reconstructed section of the mandible, with proper occlusion with the teeth of the maxilla.

### SUMMARY

A prosthetic module for a mandible bone includes a body having a form corresponding to the mandible bone and extending between a first end and a second end positioned opposite the first end. A first stem extends from the body at the first end and is configured to extend into and couple the body to a mandible bone in replacement of a resected section thereof. At least one of a second stem, a hole, or a geometry similar to a condyle-ramus-angle part of a mandible bone is located at the second end thereof.

A mandibular prosthesis includes a first prosthetic module having a body with a form corresponding to the mandible bone and extending between a first end and a second end positioned opposite the first end. The first end includes a first stem extending therefrom and configured to extend into and couple the body to a mandible bone in replacement of a resected section thereof. The second end includes one of a second stem extending therefrom or a first hole formed therein. A second prosthetic module includes a body having a form corresponding to the mandible bone and extending between a first end and a second end positioned opposite the first end. The first end includes the other of the second stem extending therefrom or the first hole formed therein and configured to be coupled to the second end of the first prosthetic module.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 shows an embodiment of a prosthesis for mandibular reconstruction according to the present disclosure wherein first and second prosthetic modules are fitted and shown in 2-dimensional view;

FIG. 2 shows an alternative embodiment of a prosthesis of the present disclosure wherein only a first prosthetic module is employed;

FIG. 3 shows the embodiment of FIG. 1 wherein ports with cone implants and are used to affix the protuberance in the corresponding bore or hole in the adjoining bone of the mandible resection modules;

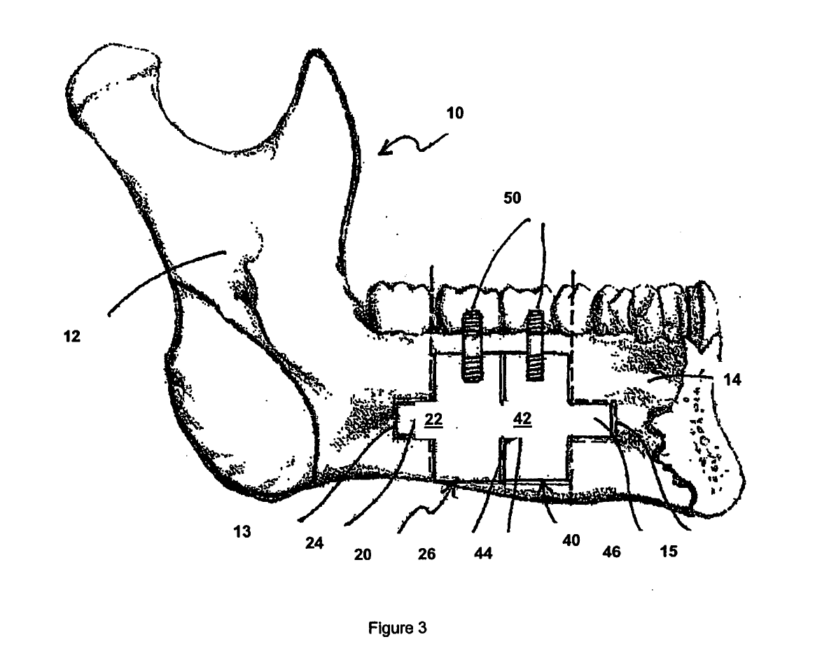

FIG. 4 shows another alternative embodiment wherein a third prosthetic module is implanted;

FIG. 5 shows yet another embodiment wherein a third prosthetic module, in combination with the second prosthetic modules bridge the resection;

FIG. 6 shows an example of the third prosthetic module of FIG. 5 wherein is shown with the surface area which are sintered being marked;

FIG. 7 shows a cross-sectional view of a resectioned mandible at the molar region (as shown in an inset photograph) proposed to be reconstructed according to our disclosure;

FIG. 8 shows two cross-sectional views, in perspective, of a resectioned mandible at the cuspid region proposed to be reconstructed according to our disclosure;

FIG. 9 shows a fourth module as an embodiment wherein two modules are shown to join to each other and to the resected bone interfaces with two prongs and two stems;

FIG. 10 shows a cross-sectional view of a distal end of a resected mandible at the molar region as shown in the inset picture; and

FIG. 11 shows a cross-sectional view of a mesial end of a resected mandible as shown in the inset picture.

### DETAILED DESCRIPTION

The following description is merely exemplary in nature and is not intended to limit the present disclosure, application, or uses.

a “module” refers to the main body of the prosthesis which may be connected to a resected mandible or to another prosthesis in a modular manner;

a “stem” is that part of the prosthesis that is inserted as a protuberance and cemented into the remaining bone of the resected mandible;

a “taper lock” is used to connect two modules without using cement, including but not limiting to Morse lock;

a “port” is a screw-threaded hole on the prosthesis;

a “cover screw” is inserted in the port before operation so soft tissues can not grow therein; and

a “cone” is screwed into the part after removing the cover screw, about 6 weeks after the operation; the cone protrudes through the gum into the mouth for the insertion of teeth later.

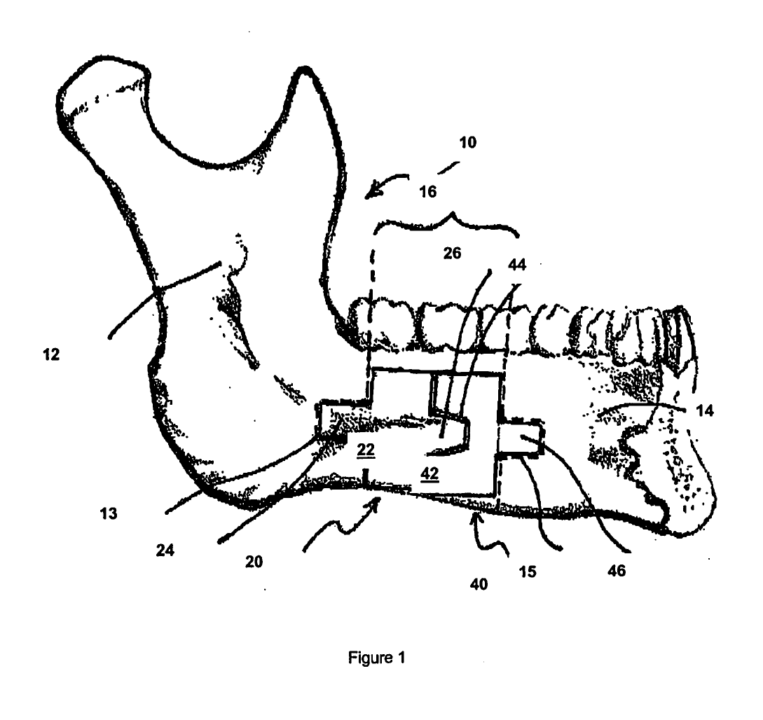

The general embodiment of our disclosure may be described with reference to FIG. 1 which shows a mandible (10) being transposed with our proposed prosthesis which comprises a first prosthetic module (20), hereinafter “module A”, and a second prosthetic module (40), hereinafter “module B”. The mandible (10) has been rendered segmental, i.e., discontinued into two segments, i.e., the condyle-ramus-angle portion (12) and the symphysis (14), due to the resected part (16) of the mandible body (16).

Module A (20) typically comprises a body (22) having a thickness or profile corresponding to the mandible body (16). A stem (24) extends from one end of the body (22) into a fitting bore (13) drilled into the condyle-ramus-angle portion (12) of the mandible. A tongue (26) provided as a taper lock is provided to extend from the other end of the module body (22).

Module B (40) typically comprises a body (42) having a thickness or profile corresponding to the mandible body (16). A hole (44), complementary to the tongue (26) of module A, is provided at one end of the body (42). A stem (46) extends from the other end of module B's body (42), such that the stem (46) fits into a bore (15) drilled into symphysis (14) portion or the other segment of the resected mandible.

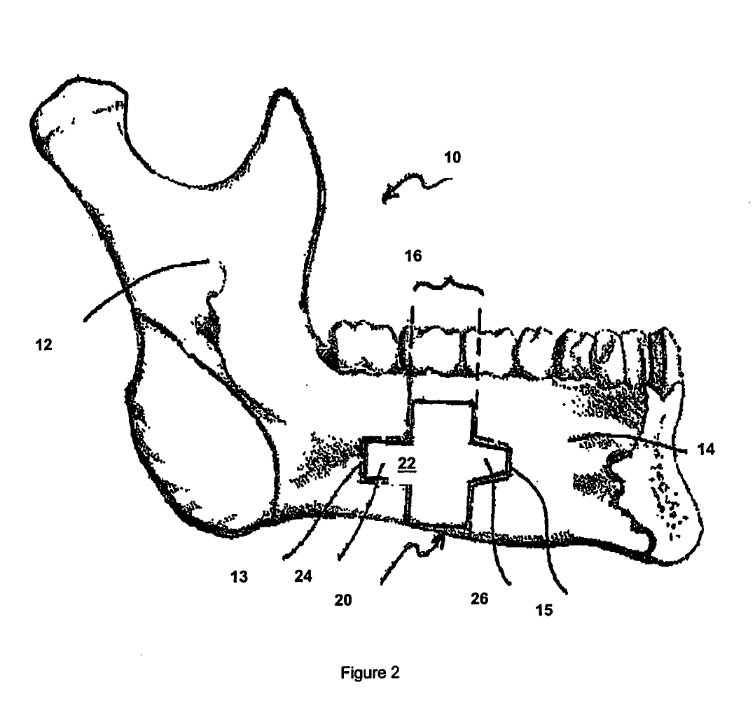

A configuration of our modular prosthesis for segmental mandibular reconstruction includes module A being used alone as shown in FIG. 2 wherein the tongue (26) is adapted to function as stem to fit into a bore on the other segment of the resected mandible. This embodiment is suitable for a short resection where the length of module A's body (22) would be sufficient to bridge the resection.

It should be noted that, in all our drawings herewith, although the joints such as the stem-in-bore (24, 13 and 46, 15) and tongue-in-hole (26, 44) are shown in different shapes or profile, the tongue (26) of module A (20) may be employed as a stem to fit into a bore (15) provided in the bone, as shown in FIG. 2, as long as the shapes of the stem (24, 46) or tongue (26) have compatible profile and gauge with each other to fit into the bore (13, 15) or hole (44).

The first prosthetic module or module A may include a body having a thickness and form which correspond to a mandible body and is about 5 mm below the gingiva, a stem extending from one end of the first prosthetic module body into a fitting bore on a first mandible bone segment cemented therein; and a first tongue extending from another end of the first prosthetic module body.

The second prosthetic module or module A may include a body having a thickness and form corresponding to a mandible body and is about 5 mm below the gingival. A groove is provided to extend into one end of said module-body to complementarily and fittingly receive a first tongue of the first prosthetic module, and a stem extending from another end of said second prosthetic module body into a fitting bore on a second mandible bone segment.

As one of the features of our modular prosthesis is to enable the prosthetic modules A (20) and B (40) to be linked to each other in repetition as needed (i.e., in multiple units of module B) to bridge the resection, the manner in which our linkage is formed, i.e., via tongue (26)-in-hole (44) means should allow for adjustments so that the linkage formed from a series of modules A and B to conform to the desired contour of the mandible resection. To enable such adjustments, it may be advantageous to provide for the protuberating parts such as the stem (24, 46) and tongue (26) be provided with a slight or gentle tapering so that it may be inserted into its corresponding hole (44) or bore (13, 15) to form a friction-tight taper lock. A Morse lock is an example of such friction tight taper lock as described in U.S. Pat. No. 5,314,479, referred to herein as an enabling technology.

In yet another aspect of the disclosure, bone cement is used to affix the protuberances in the corresponding bores or holes in the bones. An example of a suitable cement composition available commercially is Palacos G bone cement from Biomet Orthopedics, Inc.

Modules A and B are reconstructed so that about 5 mm space below the gingiva is provided for soft tissue healing. Modules A and B of our disclosure are capable of taking in cones into the metal endoprosthesis at the ports or bores so teeth or dentures can be fabricated upon them in the form of various prosthetic super structures available in the market without the need for bone as a substrate for implanting.

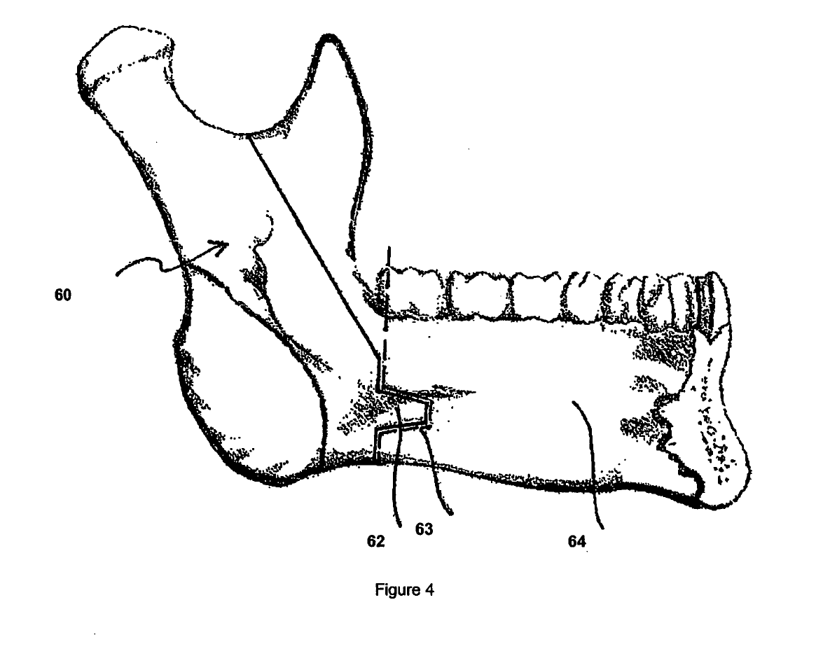

In another aspect of our disclosure, a third prosthetic module, hereinafter module C, is provided, as shown in FIG. 4. Module C (60) endeavors to replace the condyle-ramus-angle part of the mandible, and which may be used as a replacement for the anchoring of either module A or B. A tongue-in-hole element, i.e., either a tongue or a hole, may be provided at the interfacing part of the module with module A or B. The example shown in FIG. 4 illustrates the condyle-ramus-angle part of the mandible having been resected and is shown replaced with a module C. The tongue-in-hole element is shown as a tongue (62) which may plug fittingly into a bore (63) drilled onto the mandible body (64) which is not affected by the resection.

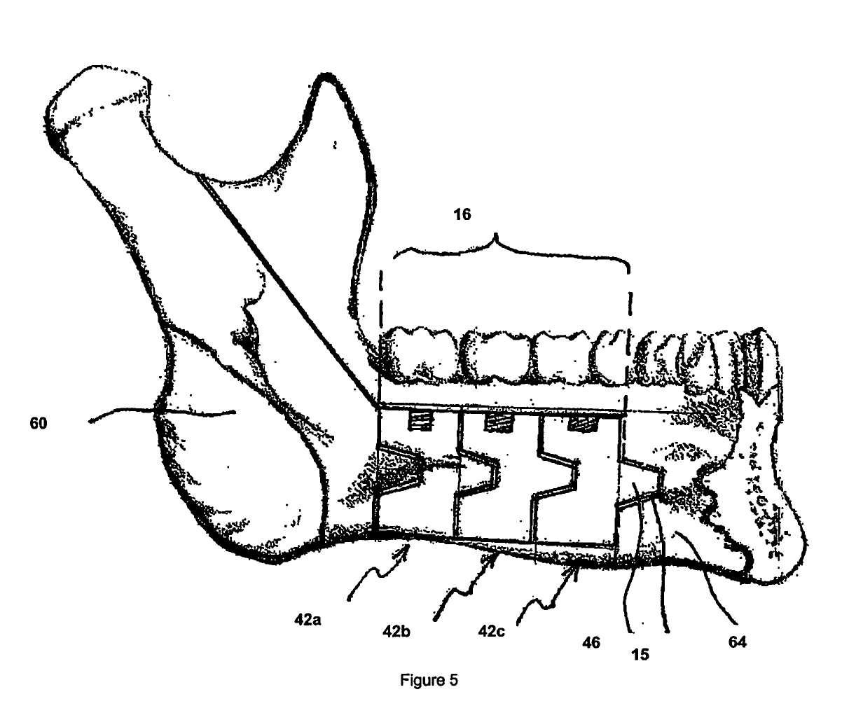

FIG. 5 shows a mandible which condyle-ramus-angle portion has been replaced by a module C and a plurality of modules B to bridge the resected part of the mandible to connect with the symphysis segment. Hence, depending on the length of each of the modules B, an appropriate number of modules B may be modularly linked end-to-end anchored at one end at module C and the other with the good, unresected symphysis segment.

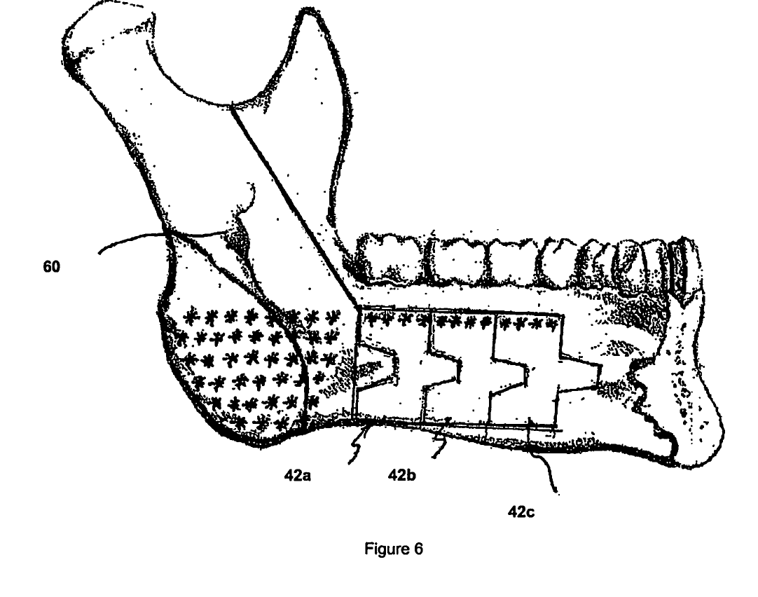

The above-described prosthetic modules are made of biocompatible material including biocompatible metal or alloys. These include using cobalt-chrome alloy or titanium which has been used successfully in many types of endoprosthesis. The prosthetic modules' shapes and configurations may be made by any one or combination of metal-casting, machining and metal-forging. The surfaces of the module may be provided a texture which facilitates muscles or tissue to re-grow and attach thereon, including bone tissues. To this end, certain methods of increasing surface area and to provide micro-projections for tissue attachment may be provided, such as sintering. Beads of the appropriate sizes may be sintered at desirable locations such as marked “*” in FIG. 6.

Other means of encouraging tissue regrowth and attachment to the module surfaces may be provided, for example, by providing porosity on the surface of the modules, mesh sheath woven of metal wires or buttons made from titanium, stainless steel and chrome cobalt as proposed in U.S. Pat. No. 4,064,567 mentioned herein as an enabling technology. Biocompatible synthetic polymers such as carbon and graphite fibres with optional metallic and ceramic fibres, bonded together by sintered polytetrafluorethylene (PTFE) have also been suggested in U.S. Pat. No. 3,992,725, and a large number of biocomposite materials referred to in U.S. Pat. No. 5,084,051, are all mentioned herein as enabling technology.

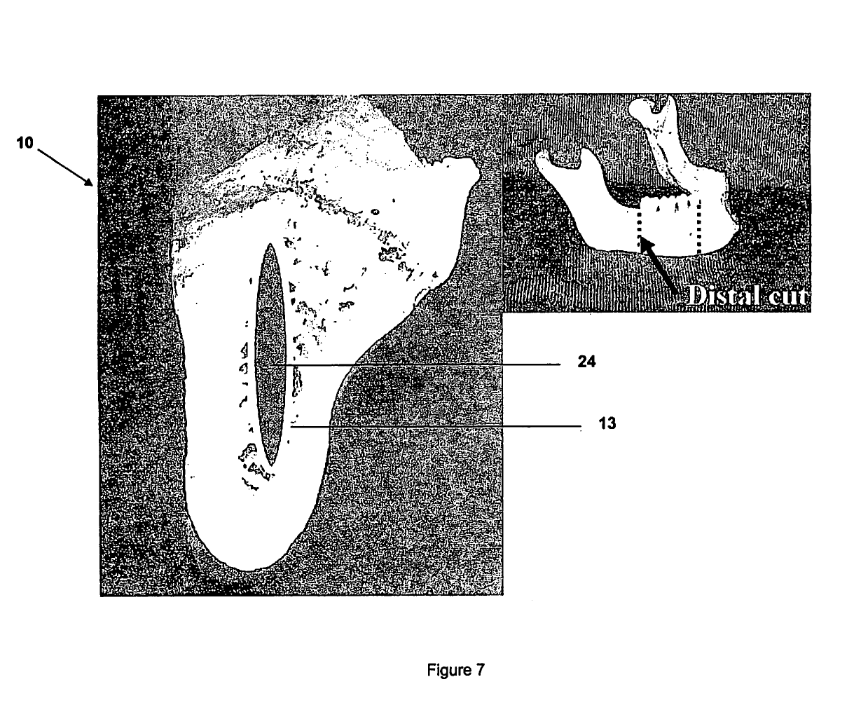

FIG. 7 shows a cross-sectional view of a resectioned mandible at the molar region, specifically at where is marked as “distal cut” shown in the inset photograph. The cross-sectional view provides an idea of the cross-sectional profile of the modules required for reconstruction purposes, including providing the bore (13) for the stem (24) extending from the module to be inserted thereinto. It should be noted that the module's upper surface should be provided 5 mm below the gingiva.

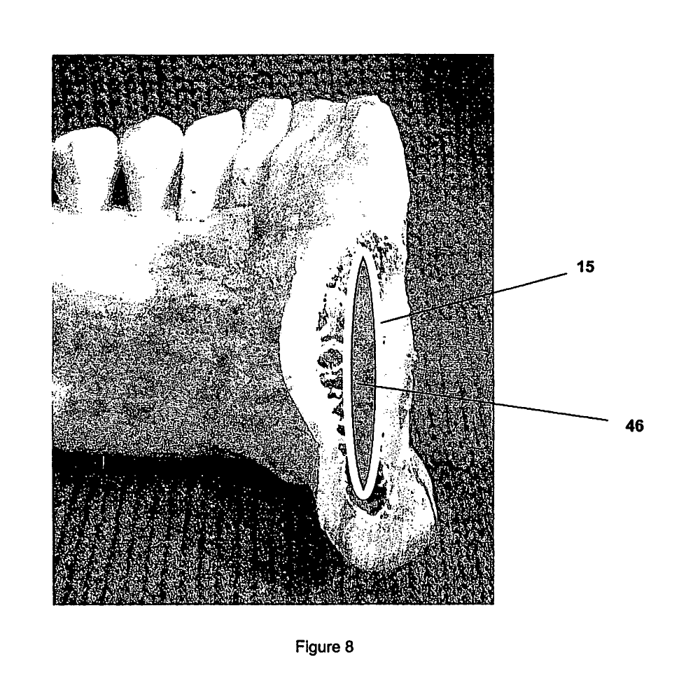

FIG. 8 shows two cross-sectional views, in perspective, of a resectioned mandible at the cuspid region, which is proposed to be reconstructed according to our disclosure. At this particular region, as shown, the module to be fabricated would have a thickness of about 11 mm at the upper portion and about 9 mm at the lower portion with about 25 mm in height.

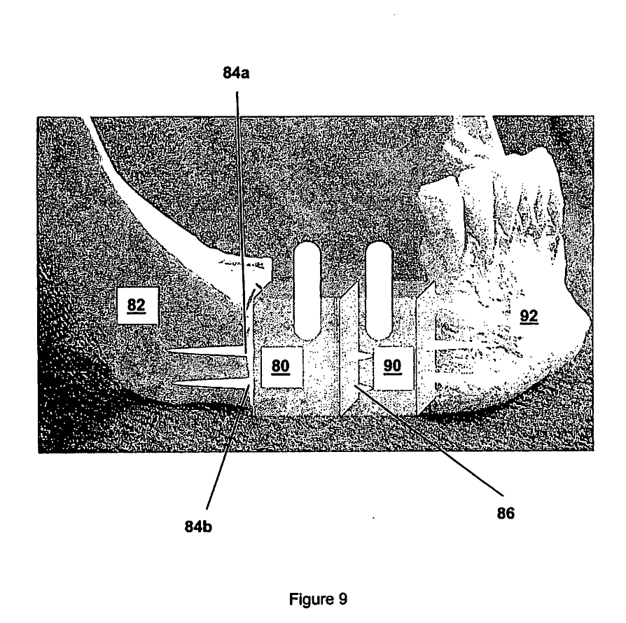

FIGS. 9 to 11 collectively show a fourth module as another embodiment of our disclosure wherein the two modules (80, 90) are shown to join to each other and to the resected bone interfaces (82) with two prongs and two stems. The pair of prongs (84a, 84b) are relatively longer than the stems (86) as the former are required to be protrude into the resected bone surfaces to form a secure tongue-in-groove joint. The stems (86) may be provided to extend into corresponding apertures on the adjoining module. Both the stems (86) and prongs (84a, 84b) may be tapered to have the Morse lock or taper locking effect.

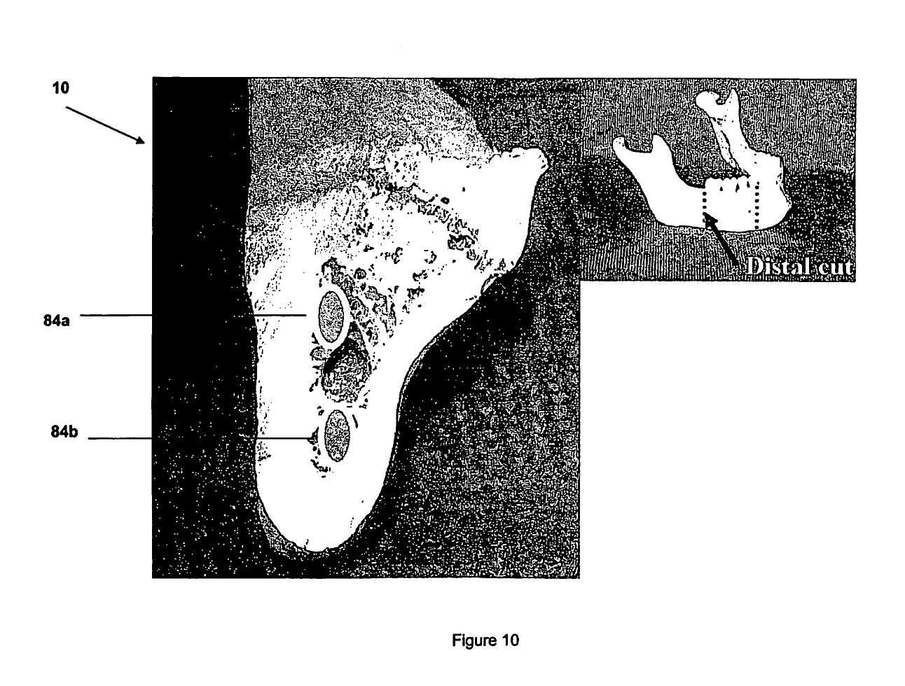

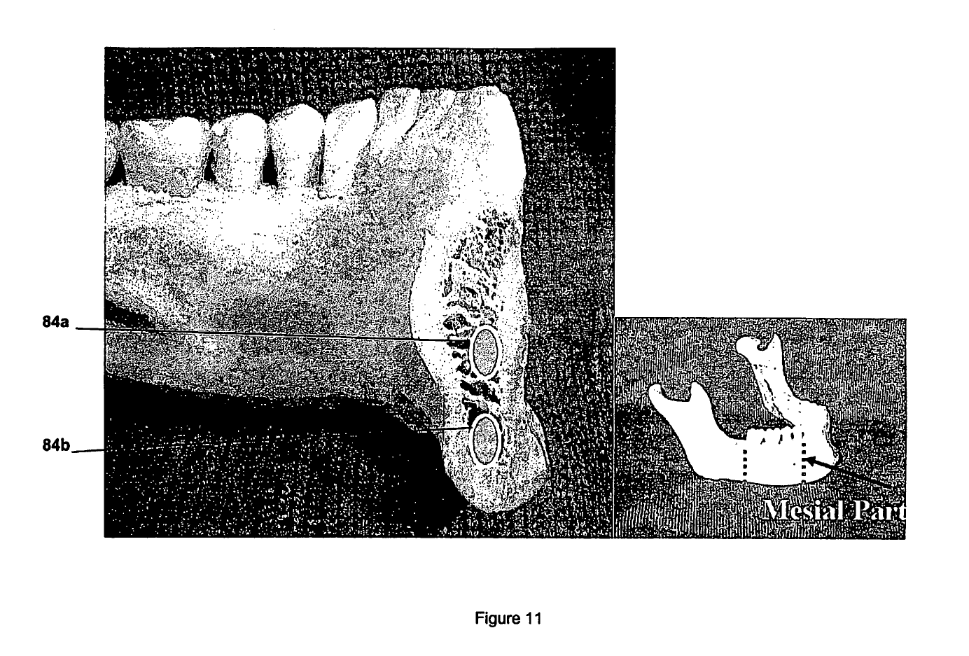

FIG. 10 shows a cross-sectional view of a distal end of a resected mandible at the molar region as shown in the inset picture. It shows the two prongs' cross-sectional view with the corresponding bores. In addition to the taper locking upon insertion into the corresponding bores, bone cement may be added and allowed to set to strengthen the tongue-in-groove joints. As shown in the inset picture, FIG. 11 shows a cross-sectional view of a mesial end of a resected mandible.

It will be appreciated that a number of the above-described features of our disclosure may be adopted modularly, modified, reconfigured, or alternatively adapted according to the required heat exchange efficiency or demands by users which are still based on the same general concept, features and working principles of the present disclosure.

These variations and alternative embodiments may be used in substitution of the aforesaid parts, components, materials, steps or processes as alternative configurations or embodiments not specifically described herein but which may still be used to effectively work the concept and working principles of this disclosure. Accordingly, they are not to be considered as departures from the present disclosure but shall be considered as failing within the letter and scope of the following claims.

What is claimed is:

1. A prosthetic module for a mandible bone comprising:
a body having a form corresponding to the mandible bone and extending between a first end and a second end positioned opposite said first end;
a first stem extending from said body at said first end and configured to extend into and couple said body to a mandible bone in replacement of a resected section thereof; and
at least one of a second stem, a hole, or a geometry similar to a condyle-ramus-angle part of a mandible bone at said second end thereof.

2. The prosthetic module of claim 1, wherein said second stem is configured to extend into and couple said body to one of the mandible bone and an adjacent module.

3. The prosthetic module of claim 1, wherein said hole is configured to receive a stem of an adjacent module.

4. The prosthetic module of claim 1, wherein said body includes a surface configured to facilitate attachment of muscle or tissue thereto.

5. The prosthetic module of claim 1, wherein said body and said first stem are formed from a biocompatible material.

6. The prosthetic module of claim 1, wherein said body has a thickness generally equal to a thickness of the resected section of the mandible bone.

7. The prosthetic module of claim 1, wherein said body has a height configured to extend below the gingiva.

8. The prosthetic module of claim 1, wherein said body is configured to receive a dental implant therein.

9. A mandibular prosthesis comprising:
a first prosthetic module including a body having a form corresponding to the mandible bone and extending between a first end and a second end positioned opposite said first end, said first end including a first stem extending therefrom configured to extend into and couple said body to a mandible bone in replacement of a resected section thereof, said second end including one of a second stem extending therefrom or a first hole formed therein; and
a second prosthetic module including a body having a form corresponding to the mandible bone and extending between a first end and a second end positioned opposite said first end, said first end including the other of said second stem extending therefrom or said first hole formed therein and configured to be coupled to said second end of said first prosthetic module.

10. The mandibular prosthesis of claim 9, wherein said second end of said second prosthetic module includes a third stem extending therefrom.

11. The mandibular prosthesis of claim 10, wherein said third stem is configured to extend into and couple said body of said second prosthetic module to the mandible bone.

12. The mandibular prosthesis of claim 9, further comprising a third prosthetic module including a body having a form corresponding to the mandible bone and extending between a first end and a second end opposite said first end, said first end of said third prosthetic module including one of a fourth stem extending therefrom and a second hole formed therein, said second end of said second prosthetic module including the other of said fourth stem extending therefrom and said second hole formed therein, said first end of said third prosthetic module and said second end of said second prosthetic module configured to be coupled to one another.

13. The mandibular prosthesis of claim 9, wherein said second end of said second prosthetic module includes a geometry generally similar to a condyle-ramus-angle part of a mandible bone.

14. The mandibular prosthesis of claim 9, wherein one of said bodies includes a surface configured to facilitate attachment of muscle or tissue thereto.

15. The mandibular prosthesis of claim 9, wherein said bodies of said first and second prosthetic modules and said first stem are formed from biocompatible materials.

16. The mandibular prosthesis of claim 9, wherein said bodies of said first and second prosthetic modules each have a thickness generally equal to a thickness of a resected section of the mandible bone.

17. The mandibular prosthesis of claim 9, wherein said bodies of said first and second prosthetic modules each have a height configured to extend below the gingiva.

18. The mandibular prosthesis of claim 9, wherein at least one of said bodies of said first and second prosthetic modules is configured to receive a dental implant therein.

19. The mandibular prosthesis of claim 9, further comprising a cement disposed between said first end of said first prosthetic module and the mandible bone, thereby coupling said first prosthetic module to the mandible bone.

20. The mandibular prosthesis of claim 9, further comprising a cement disposed between said second end of said first prosthetic module and said first end of said second prosthetic module, thereby coupling said first and second prosthetic modules to one another.
